(19) United States
(12) Patent Application Publication
(10) Pub. No.: US 2024/0207171 A1
(43) Pub. Date: Jun. 27, 2024

(54) BIODEGRADABLE COMPACTED FORMULATIONS AND METHODS OF USE AND MANUFACTURE THEREOF

(71) Applicant: Purdue Research Foundation, West Lafayette, IN (US)

(72) Inventors: Andrew David Otte, West Lafayette, IN (US); Kinam Park, West Lafayette, IN (US)

(21) Appl. No.: 18/538,022

(22) Filed: Dec. 13, 2023

Related U.S. Application Data
(60) Provisional application No. 63/435,260, filed on Dec. 25, 2022.

(51) Int. Cl. A61K 9/00; A61K 31/485

(52) U.S. Cl. CPC A61K 9/0024; A61K 31/485

(57) ABSTRACT

The present invention generally relates to formulations comprising a drug and biodegradable polymers that are compacted mechanically from a physical mixture to disrupt interconnected open channels for substantially longer drug release than non-compacted counterpart formulations and methods of use and manufacture thereof.

### RELATED APPLICATION

This application claims benefit of U.S. provisional patent application No. 63/435,260, filed Dec. 25, 2022, the contents of which is incorporated by reference herein in its entirety.

### FIELD OF INVENTION

The invention generally relates to biodegradable compacted formulations and methods of use and manufacture thereof.

### BACKGROUND

Opioid use disorder (OUD) has become an epidemic in the United States, and accessible OUD treatments are urgently required. Untreated OUD, a chronic brain disease, has a serious cost to people, their families, and society. In 2022 alone, more than 100,000 people died due to drug overdose. Each year, opioid overdose, misuse, and dependence account for more than $100 billion in healthcare costs, criminal justice costs, and lost productivity. Mortality related to opioid use disorder (OUD) has been reduced by opioid agonist therapy (OAT), including buprenorphine (buprenorphine) and methadone. Methadone is a synthetic, full agonist of the μ-, κ-, and δ-opioid receptors, causing a higher risk for respiratory depression and higher comorbid mental disorders than buprenorphine. One of the advantages of using a partial agonist in therapy for OUD is induction time into therapy. Due to the 7-10 days of opioid abstinence required to administer VIVITROL (1 month extended-release naltrexone—pure competitive opioid antagonist) due to the delayed release of the drug, initiation is more complex than oral buprenorphine-naloxone. Furthermore, working on improvement of treatment retention was noted for both VIVITROL and oral buprenorphine-naloxone. A recent retrospective electronic health records study found that 50-75% of patients who began a buprenorphine treatment, left within three months.

The buprenorphine products approved by the U.S. Food and Drug Administration (FDA) for the treatment of OUD include oral and injectable formulations. Oral sublingual tablets and films have considerable diversion of the medications and risks of nonadherence. Extended-release depot injection of buprenorphine can prevent diversion, daily fluctuations in plasma concentrations, poor daily adherence, medication misuse, accidental poisoning in children, and possibly provide improvements in treatment retention.

### SUMMARY

The invention recognizes that providing additional and longer acting buprenorphine-based formulations for patients and physicians is one potential simple method that can provide a means of improving treatment retention. Our research on long-acting formulations based on biodegradable polymers, such as poly(lactide-co-glycolide) (PLGA), and understanding of the drug release mechanisms provides us with a new, potentially breakthrough technology that can be readily applicable to long-acting formulations requiring a high drug loading and controlled release kinetics. Our new compacted single-rod method described herein requires control of fewer processing variables, that is readily scalable, and with a potentially lower cost than microparticles. The rod platform can overcome typical problems associated with other formulations, such as PLGA microparticle formulations with poor drug loading and high initial burst release if not formulated correctly. Compacted rods provide an ideal vehicle for extremely high drug loads, readily scalable manufacturing, and longer durations of therapy. The increased microstructure density of the compacted rods allows slower water uptake kinetics, ultimately resulting in slower drug release.

To that end, and in certain aspects, the invention provides biodegradable, implantable formulations comprising a drug and one or more biodegradable polymers, wherein the formulation is essentially free of pores.

In other aspects, the invention provides methods of treating a subject with an opioid abuse disorder, that involve implanting in a subject a biodegradable implantable formulation comprising a drug that treats opioid abuse and one or more biodegradable polymers, wherein the formulation is essentially free of pores.

In other aspects, methods of making a biodegradable formulation that is substantially free of pores that involve providing a mixture of a drug and one or more biodegradable polymers; compacting the mixture with sufficient mechanical force to remove surface pores and a substantial amount of internal pores; and applying heat to sustainably remove remaining internal pores, thereby producing a biodegradable formulation that is substantially free of pores.

In certain embodiments of the formulations and methods herein, the biodegradable formulation comprises about 40 to 80% by weight of drug. In certain embodiments of the formulations and methods herein, the biodegradable formulation comprises about 50 to 70% by weight of drug.

In certain embodiments of the formulations and methods herein, the biodegradable formulation comprises about 20 to 60% by weight of biodegradable polymer. In certain embodiments of the formulations and methods herein, the biodegradable formulation comprises about 30 to 50% by weight of biodegradable polymer. In certain embodiments of the formulations and methods herein, the biodegradable polymer is a PLGA polymer, Poly(D,L-lactide), Poly(ε-caprolactone), Polyhydroxybutyrate, Polyanhydrides, Polyorthoesters, or combinations of any of these. In certain embodiments of the formulations and methods herein, the biodegradable formulation provides sustained release of drug for about 90 days or longer. In certain embodiments of the formulations and methods herein, the biodegradable formulation a surface of the biodegradable formulation is free of pores.

In certain embodiments of the formulations and methods herein, the drug is buprenorphine. In certain embodiments of the formulations and methods herein, the buprenorphine is of the free base (FB) form, salt form, or mixtures thereof.

### BRIEF DESCRIPTION OF THE DRAWINGS

FIG. 1 shows extrusion and crimping processes of making buprenorphine-loaded compacted rod formulation.

FIG. 2 shows (From bottom to top) powder x-ray diffraction patterns of Buprenorphine hydrochloride (Bup HCl) from the Cambridge Structural Database (CSD), Buprenorphine HCl tested as received, Buprenorphine free base (BUP FB) from the CSD, and Buprenorphine FB prepared.

FIG. 3 shows in the left column a cross-sectional and longitudinal image of an implant post drying; middle column, cross-sectional and longitudinal image of an implant post compaction; and right column, end of implant post heat annealing.

FIG. 4 is a graph showing a prototype rod buprenorphine rod formulations prepared with Evonik 858 and Ashland 8507E polymers. *Resomer 858 Compaction release started one day later than Resomer 858 with no compaction.

FIG. 5 is a graph showing in vitro release of compacted rods prepared from buprenorphine:PLGA physical mixtures of 3 different PLGA 85:15 IV values compared to Probuphine.

FIG. 6 is a graph showing in vitro release of buprenorphine FB raw powder and 100% buprenorphine FB compact.

FIG. 7 is a graph showing the in vitro release rate of buprenorphine from a dosage form with varying concentrations of the buprenorphine FB and HCl.

FIG. 8 is a graph showing a comparison of the in vitro release profiles of a 70% buprenorphine drug loaded implants compacted at 500 PSI, 250 PSI, or via the ejection force only.

FIG. 9 is a set of graphs showing comparison of in vitro release of 50%, 60%, and 70% loading of buprenorphine FB.

### DETAILED DESCRIPTION

The present disclosure relates to injectable, controlled-release (or sustained-release) compacted formulations comprising buprenorphine and a biodegradable polymer such as a PLGA polymer, Poly(D,L-lactide), Poly(ε-caprolactone), Polyhydroxybutyrate, Polyanhydrides, Polyorthoesters, or combinations of any of these.

It is highly useful to understand the drug release mechanisms to develop the formulations having the intended drug release profiles. Trial-and-error approaches, typically used in prior formulation development, provide no scientific basis to understand and improve the drug release kinetics. In particular, the initial burst release is ubiquitous in the current FDA-approved formulation, and its causes are not clearly understood while various theories have been proposed, and thus, is difficult to prevent.

Our recent studies indicate that the initial burst release is due to rapid absorption of water into PLGA formulations and subsequent rapid release of the loaded drug (Park et al., Injectable, long-acting PLGA formulations: Analyzing PLGA and understanding microparticle formation, J. Controlled Rel. 304: 125-134, 2019; Park et al., Potential roles of the glass transition temperature of PLGA microparticles in drug release kinetics, Mol. Pharm. 18: 18-32, 2021; Otte et al., The impact of post-processing temperature on PLGA microparticle properties, Pharm. Res. 2023, https://doi.org/10.1007/s11095-023-03568-z). The initial burst release is followed by rearrangement of PLGA polymers at 37° C., close to their glass transition temperatures (T_{g}), forming a PLGA membrane on the surface controlling drug release at the steady state. The fast absorption of water upon exposure of PLGA formulations to water is due to the presence of interconnected pores in the formulations, which are formed as a result of removing the solvent used in making the PLGA formulations. Thus, an important factor to prevent the initial burst release is to abolish the interconnected pores. Eliminating the interconnected pores in a biodegradable matrix is an important aspect of the new formulations.

Furthermore, the duration of buprenorphine release from PLGA 50:50 microparticles with only ≤5% of the total weight is only 3 days. SUBLOCADE (buprenorphine extended release, Indivior) is an in situ forming implant with 20% buprenorphine loading in PLGA 50:50, releases the drug for 1 month. Use of in situ forming gel PLGA-PEG-PLGA triblock copolymer for delivery of buprenorphine for 1 month (PLGA 75:15) but the buprenorphine loading was very low at 1% w/w. For the ≥3-month buprenorphine release formulations, PLGA polymers need to have the L:G ratio of 75:25 to 90:10 to provide a balance of release and degradation profile within a clinically relevant time frame. PLGAs within this L:G ratio window degrade slowly enough to provide release for ≥3 months, a lower burst release, but also degrade within 3-10 months.

The injectable buprenorphine formulations are usually configured into one of the three delivery systems: injectable in situ forming implants (ISFI, e.g., SUBLOCADE), microparticles (MPs), and solid PLGA implants (e.g., PROBUPHINE (buprenorphine implant, Titan Pharmaceuticals)). ISFIs have not been demonstrated to be clinically useful for ≥3-month buprenorphine delivery to date. Microparticle formulations can certainly deliver buprenorphine for 3 months or longer, through finding the optimum formulation variables and processing conditions for scale-up manufacturing. Microparticle formulations can encounter issues during scale-up and control over the multiple parameters that dictate the drug loading and release.

Our research on long-acting PLGA formulations and understanding on the drug release mechanisms provides us with a new, potentially breakthrough technology that can be readily applicable to long-acting formulations requiring a high drug loading and controlled release kinetics. Our new compacted single-rod method described herein requires control of fewer processing variables, that is readily scalable, and with a potentially lower cost than microparticles. The rod platform can overcome typical problems associated with other formulations, such as PLGA microparticle formulations with poor drug loading and high initial burst release if not formulated correctly. Compacted rods provide an ideal vehicle for extremely high drug loads, readily scalable manufacturing, and longer durations of therapy. The increased microstructure density of the compacted rods allows slower water uptake kinetics, ultimately resulting in slower drug release. Key design parameters for ≥3-month delivery formulations are described below.

The compaction of drug-PLGA mixture powders include the compaction step that results in reduction in volume by displacing the gaseous phase and subsequent particle elastic/plastic deformation and consolidation (assembly into a single object), or fusion. The drug-PLGA mixture can include other pharmaceutical excipients such as a binder. The main purpose of compaction is to displace gaseous phase and minimize the pores or open channels to control the drug release and extend the drug release duration for several months. The optimal compaction requires important selection of PLGAs with the right particle size and density for desired dimensions of punch and die. Other polymers include but are not limited to Poly(D,L-lactide), Poly(ε-caprolactone), Polyhydroxybutyrate, Polyanhydrides, Polyorthoesters, or combinations of any of these.

The commercial failure of 6-month PROBUPHINE formulation is in large part due to surgical insertion of 4 (and up to 5) implants, followed by the surgical removal after 6 months. PROBUPHINE was made by hot-melt extrusion of buprenorphine hydrochloride (HCl) and ethylene-vinyl acetate (EVA) copolymer blend to form a fiber with a diameter of 2.4 mm. The extruded non-biodegradable fiber was cut into implants of 26 mm in length. The implants were washed in 95% ethanol at room temperature for 30 min to remove surface drug and thus minimize the initial release of buprenorphine. The washed implants were dried (air dried at room temperature for 30 min, then forced air at 40° C. for 1 hour followed by vacuum drying at 30° C. for 24 h) to remove residual ethanol. This process resulted in numerous interconnected pores, despite the fact the implants were washed with ethanol.

Our long-acting buprenorphine formulations described here provide a novel and valuable improvements over prior art formulations. These improvements include the following. The polymer used is biodegradable so that surgical removal at the end of the product use is not necessary. In the event surgical removal is required due to a desired therapy discontinuation, the product may be able to be removed early. The PLGA type is judiciously selected for controlling the drug release duration and kinetics. The PLGA formulation is substantially devoid of interconnected pores that can minimize the initial burst release and maintain desired stead-state release kinetics for longer periods of time. The formulation is preferably provided in a single configuration and the drug loading is high enough for administering only one implant. The formulation is preferably formulated in such a fashion that it can be manufactured under GMP conditions in a cost-effective and timely manner.

PROBUPHINE is a non-erodible implant consisting of ethylene-vinyl acetate (EVA) copolymer. To fabricate implants, buprenorphine is dry blended with EVA copolymer, followed by extrusion to form a fiber ˜2.4 mm in diameter, and cut into implants 26 mm in length. A washing step with ethanol is performed to remove surface buprenorphine; although, the percent amount of buprenorphine lost in this step is not certain. Release from non-erodible implants is predominantly dictated by the surface area, the rate of drug dissolution, and the drug diffusion through the polymeric matrix. The EVA implant is highly porous along the z-axis. As the implant is non-erodible, porosity is required so water can imbibe the system leading to buprenorphine release.

Calculation of the buprenorphine dose for a long-acting formulation requires understanding of the minimum effective concentration (Cₘᵢₙ) for clinical efficacy. Various studies on buprenorphine pharmacokinetic studies in humans have shown that the Cₘᵢₙ is 0.1 ng/ml with the aim of 0.5˜0.7 ng/mL. This target range matches with the plasma concentrations achieved by Probuphine (4˜5 implants). The mean steady-state plasma buprenorphine concentration over 6 months was approximately 0.5˜1 ng/mL. PROBUPHINE is effective in treating OUD and clinically similar to those receiving sublingual buprenorphine/naloxone, and is indicated for the maintenance treatment of opioid dependence in patients who have achieved and sustained prolonged clinical stability on low-to-moderated doses of a transmucosal buprenorphine product. The effective buprenorphine concentrations in animals are about the same, such as common marmosets, dogs, mice, and rats. FDA has released “Opioid Use Disorder: Developing Buprenorphine Depot Products for Treatment,” a guidance document encouraging widespread innovation and development of new buprenorphine-based treatments for OUD.

PROBUPHINE delivers 296.8 mg buprenorphine for 6 months. Therefore, for formulations delivering buprenorphine for 3 months, for example, 150 mg buprenorphine may be sufficient to maintain the Cₘᵢₙ for 3 months. However, the 3-month dose can be significantly reduced if the initial burst release occurring hours after administration for a few days can be prevented or reduced. Surprisingly, such dose can be used to extend the duration longer than 3 months. Furthermore, when the Norvex microparticle formulation was tested in humans, 1.5 mg/day was established as a reasonable starting dose by the FDA. Thus, 150 mg for 3-month formulation provides the initial realistic target dose.

The compacted single-rod formulations have a high buprenorphine loading with controllable drug release kinetics. Owing to the ‘simpler’ processing steps (e.g., no solvent extraction in aqueous media) than microparticle based systems, the scale-up production as similar systems across a number of different industries is already present. This new compacted single-rod formulation provides an additional, longer acting treatment option for physicians and patients alike. Such increased access to buprenorphine medication for ≥3 months by a single administration provide one of the most effective and important ways to treat OUDs.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Radial Compaction of Single-Rod Formulation to Control the Drug Release Kinetics

Developing ≥3-month buprenorphine release using a 150 mg dose or greater requires preventing the initial burst release to avoid unnecessary waste of the drug and controlling the steady-state release kinetics. Although the initial burst release is due to the convective water absorption into PLGA formulations through interconnected channels, preventing the formation of interconnected pores in PLGA formulations is extremely difficult. This is because they form as a result of removing water and solvent and the PLGA polymers cannot be condensed. The best way to remove interconnected channels is by compacting PLGA formulations, but it is impractical for in situ forming implants (ISFIs) and microparticles (MPs), particularly from a manufacturing standpoint. A new method of compaction is developed for solid implants.

Compaction of rod-shaped formulations was done using a radial crimper (available from Blockwise.com) with varying forces. The diameter of the compacted rods can be varied. For example, a rod of 4 mm diameter can be compacted to 2 mm using the crimper eliminating inner empty spaces. Radial compaction may expand the rod horizontally, and thus, the crimper can be equipped with barriers at both ends to prevent horizontal expansion. FIG. 1 describes an overall approach of manufacturing compacted single-rod buprenorphine formulation.

### Example 2: Preparation of Buprenorphine Free Base

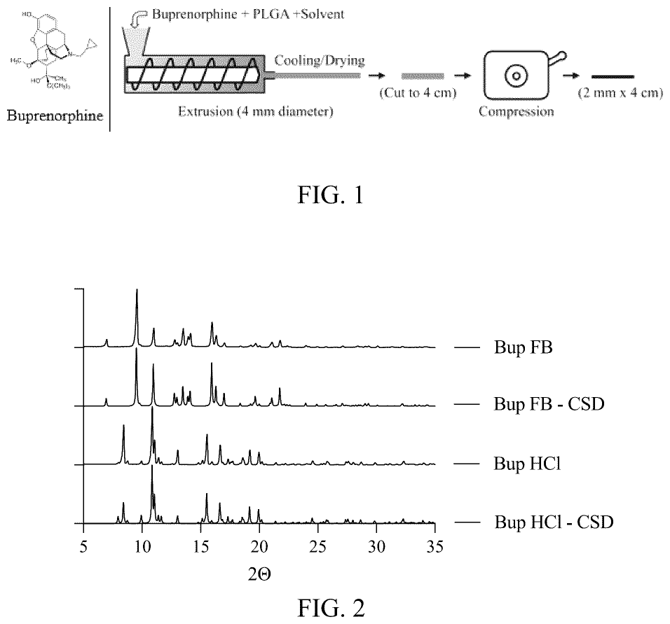

Buprenorphine free base (FB) was created by dissolving buprenorphine HCl in water with 3% v/v methanol to obtain a 1.3% solution. Buprenorphine FB was precipitated with excess NaOH. Dichloromethane at a 13% v/v ratio was then added to dissolve the precipitated buprenorphine, and subsequently a liquid-liquid extraction was performed to remove the dichloromethane-buprenorphine solution. The dichloromethane was subsequently removed via vacuum over 48 hours, resulting in buprenorphine free base. The FB and HCl salt forms were compared to the Cambridge Structural Database (CSD) forms. FIG. 2 illustrates the Powder X-Ray Diffraction patterns from the CSD compared to the HCl and FB forms used in the following examples.

### Example 3 Solubility of Buprenorphine FB

The solubility of buprenorphine FB was determined in PBS with 0.05% Tween 20 (PBST) and 0.5% w/v sodium dodecyl sulfate (SDS) in water. The solubility was determined by placing excess buprenorphine FB into the aforementioned media at 37° C. and shaken at 40 RPM for 48 hours. After 48 hours, a sample was taken and centrifuged for 15000 RPM for 5 mins, supernatant was taken, diluted with the same medium, and then the concentration was determined via High performance liquid chromatography. The solubility values are summarized in Table 1.

TABLE 1
Solubility of Buprenorphine FB in PBS, PBST, and 0.5% SDS

| | PBST | 0.5% SDS |
|---|---|---|
| Buprenorphine FB | 0.0088 mg/mL | 0.336 mg/mL |

### Example 4: Preparation of PLGA Powder

PLGA particles were prepared to act as a controlled release agent for the implantable rod. 25 g of PLGA 85:15 (Corbion; IV 0.74, 0.87, or 0.95 dL/g) or 75:25 (Durect; IV 0.55-0.75 or 0.8-1.2 dL/g) were dissolved in 80 g Dichloromethane. A 1% polyvinyl alcohol (PVA) solution was prepared in MQ water to act as an emulsifier in the continuous phase. The PLGA discontinuous phase was pumped through a Cole-Parmer gear pump at a rate of 100 mL/min and the continuous phase was pumped at a rate of 300 mL/min via Cole-Parmer gear pump through a Silverson L4R in-line homogenizer assembly. Microparticles were extracted in 15 L of a 0.1% PVA solution at 22° C. for 24 hours. Microparticles were collected between a 150 μm and 25 μm sieve and vacuum dried for 48 hours.

### Preparation of Fine Buprenorphine Powder

Buprenorphine FB was ground by hand with a mortar and pestle. The ground material was then passed through a sieve, either 63, 106 or 150 μm for a certain maximum particle size and collected for use. See Table 2.

TABLE 2
Characterization of Buprenorphine and PLGA powders.

| | d₁₀ (μm) | d₅₀ (μm) | d₉₀ (μm) |
|---|---|---|---|
| Buprenorphine FB <150 μm | 7.1 | 37.6 | 81.5 |
| Buprenorphine FB <106 μm | 6.2 | 23.1 | 43.8 |
| Buprenorphine FB <63 μm | 5.9 | 23.4 | 47.5 |
| Corbion 85:15 (0.74 dL/g) | 20.9 | 58.2 | 95.3 |
| Corbion 85:15 (0.87 dL/g) | 29.1 | 68.8 | 119.5 |
| Corbion 85:15 (0.95 dL/g) | 45.7 | 87.4 | 134.4 |
| Durect 75:25 (0.8-1.2 dL/g) | 39.3 | 86.8 | 138.2 |
| Durect 75:25 (0.55-0.75 dL/g) | 32.7 | 63.6 | 100.4 |

### Example 5: Biodegradable, Compacted Buprenorphine Rods from Solvent System

To fabricate biodegradable rods, a solution of PLGA (85:15, Ashland 8509E, IV 0.6-0.8 dL/g, M_{w}=88 kDa or Evonik 858S, IV=1.3-1.7 dL/g, M_{w}=148 kDa) in dichloromethane was prepared at 20% (8507E) or 18% (858S) w/w concentration, and buprenorphine was added at a 1:1 ratio to PLGA, resulting in a 50% drug load. The lower concentration of 858S was used due to the higher molecular weight and resultant higher viscosity. Buprenorphine crystals of less than 106 μm were used. The suspension was mixed vigorously on a shaker for 15 min. The solution was then injected into ¼″ ID platinum coated silicon tubing via a syringe and dried under vacuum to remove dichloromethane. When utilizing an extruder, the solvent content can be significantly decreased, or eliminated entirely to enable sufficient mixing of the buprenorphine crystals in the polymer matrix, resulting in a lower initial porosity than obtained here. After drying, rods were heated to 80° C. and compacted radially. Next, implants were cut and analyzed for their in vitro release.

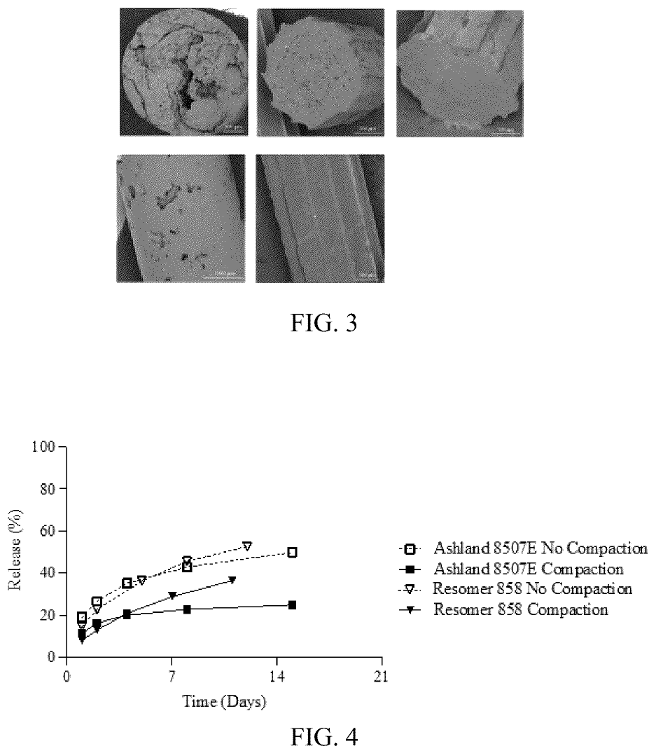

One of the advantages of using small amounts of solvent, is the intra-polymer swelling and resultant inter-polymer chain entanglement that occurs due to said solvent/swelling. Without solvent present, large amounts of shear and relatively high temperatures may be required. The final compaction process is important to minimize any porosity formed due to the solvent evaporation coupled with any incomplete consolidation during the extrusion process. FIG. 3 shows the images of the implants before and after compaction. Large pores are readily observed on the surface of the rod and cross-sectionally for implants that were not compacted (FIG. 3, left column). Post compaction (FIG. 3 middle column), nearly all surface pores were removed; although, some pores still remained visible cross-sectionally. These cross-sectional pores were closed via temperature annealing (FIG. 3, right column).

The in vitro release profiles of the implants (FIG. 4) were characterized using the same methodology as Probuphine. The diameter of the rods prepared were much smaller than that of the Probuphine formulation. Furthermore, the drug loading was also lower than Probuphine (50% vs 75%, respectively).

As the dimensions increase in size, the diffusional pathlength also increases, thus slowing drug release (coupled with a lower surface area to volume ratio). Therefore, the drug loading can be improved to higher than 50% to account for the slower kinetics as the volume is increased closer to a 2-3 mm diameter and 3.5-4 mm length. Furthermore, while the Resomer 858S showed a slightly faster release rate, this might be due to the increased initial porosity due to the higher initial solvent content, demonstrating the importance of both formulation and processing parameters.

The in vitro release data illustrates that the rods can be prepared with a biodegradable polymer, at high buprenorphine loadings, and with a release rate that strongly correlates to Probuphine. One of the most important features of this formulation and manufacturing process, is that the implementation into clinical trial manufacturing will be easier than other types of injectable long-acting formulations. The use of this in vitro method, based upon the representative performance of the Probuphine implant in vivo, has established the preparation of ≥3-month buprenorphine biodegradable single-rod formulations.

### Example 6: Biodegradable, Compacted Buprenorphine Rods from a Physical Mixture

A physical mixture of buprenorphine and PLGA was used to fabricate biodegradable buprenorphine rods. While processing with solvents has advantages, the disadvantages of using solvents include: (i) adequate removal of solvent is required, (ii) the removal of the solvent can induce macroscopic changes, such as an increase in porosity due to the extraction of the solvent from the implant, (iii) and solvent can induce unintended crystallinity changes in the loaded buprenorphine. Therefore, additional means of producing biodegradable buprenorphine rods without solvents were developed.

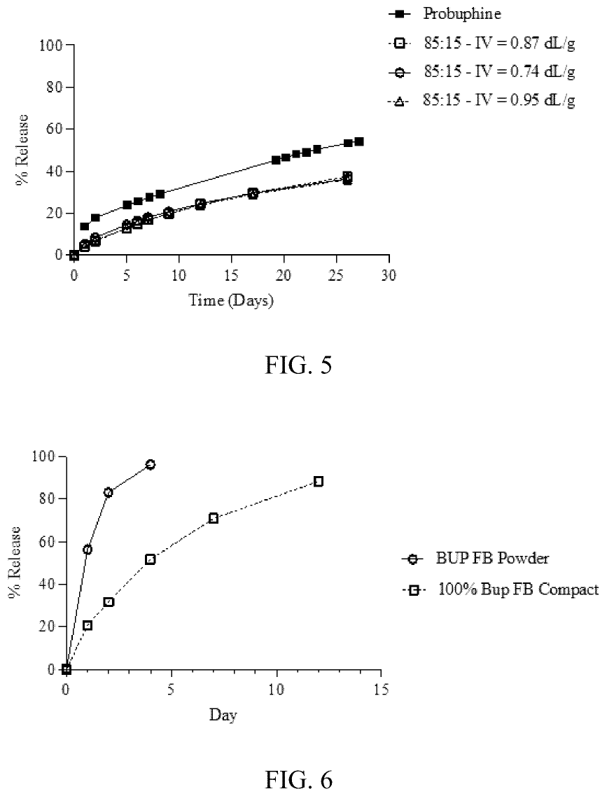

A 50:50 buprenorphine:PLGA physical mixture of 1.2 g was prepared in a 20 mL scintillation vial of each grade of Corbion polymer and mixed with a Resodyn acoustic mixer for 5 minutes. Buprenorphine used was <150 μm. ˜550 mg of the blend was filled into a Teflon die of ⅛″ and subsequently placed in an oven at ˜85° C. for 30 min. The die was removed from oven and immediately compacted by hand with aluminum plate and ⅛″ steel rod as punch. All 3 grades of polymer were compacted simultaneously to normalize compaction force across grades. Rods were allowed to cool to room temperature overnight and ejected with an arbor press. Cross-sectioned views of buprenorphine:PLGA rods prepared from physical mixture showed smooth side walls with minimal pores noted on the surface. FIG. 5 illustrates the in vitro release profiles of buprenorphine from the rods made of Corbion PLGAs with different molecular weights.

Probuphine was discontinued from the market in October 2020. For qualitative comparison purposes, the in vitro release profile from U.S. Pat. No. 7,736,665B2 (Patel, Rajesh A. and Bucalo, Louis R. Implantable polymeric device for sustained release of buprenorphine, U.S. Pat. No. 7,736,665B2, 2010) was digitized (PlotDigitizer) and converted to a percent release profile, based upon a loading per rod of 80 mg buprenorphine HCl (Probuphine (buprenorphine) implant for subdermal administration, Package Insert, Braeburn Pharmaceuticals, CIII 2016). FIG. 5 also illustrates the release of a Probuphine rod in 50 mL of 0.5% sodium dodecyl sulfate at 37° C. under orbital shaking, with samples taken and completely changing the release medium with fresh medium.

The probuphine in vitro release bears similarities to that disclosed in Titan Pharmaceuticals US Published Patent Application No. US 2016/0367477 A1. Probuphine release plotted is from a single rod implant (4-5 are given/dose). The Corbion data plotted is a fraction (˜⅛- 1/10) of a ˜500 mg implant. Thus, release hypothetically will be slightly slower from a “whole” implant compared to plotted data as surface area will decrease. Fabricating buprenorphine rods from a physical mixture of buprenorphine:PLGA produces implants that impart controlled release of buprenorphine for months.

### Example 7: Buprenorphine Free Powder and 100% Buprenorphine Implant

Free buprenorphine FB powder (<150 μm) was placed into the release media to test the in vitro release characteristics of the free drug. 500 mg buprenorphine FB powder (<150 μm) was placed into a ⅛″ die and placed in an oven at 100° C. for 30 minutes. The die containing powder was then removed from the oven and compacted manually with an aluminum plate and a ⅛″ steel rod as a punch. The formed rod was then ejected from the die and allowed 24 hours to equilibrate to room temperature prior to in vitro release testing. FIG. 6 illustrates the in vitro release of buprenorphine powder and in vitro release of a 100% buprenorphine compacted rod. The buprenorphine powder completely dissolves in ˜4 days and the implant lasts slightly longer than ˜12 days. The 100% buprenorphine rods broke apart during the in vitro release experiments, which could alter the release rate.

### Example 8: Implantable Rods Prepared from Physical Mixtures of HCl and FB Forms

Buprenorphine can exist as a free base (FB) form or a salt form (e.g., HCl salt). Incorporation of the two different forms of buprenorphine into the dosage form may affect the overall release rate of the implant based upon the solubility and dissolution rate differences of the two solid-state forms. By selecting an appropriate ratio of HCl:FB forms of buprenorphine and incorporating into said implant the release rate and/or duration of release may be modified.

In this example, buprenorphine was loaded in the dosage form as the HCl, FB, or a combination of both forms. Implants containing a total of ˜70% w/w buprenorphine were prepared with 75:25 Durect PLGA IV 0.55-0.75 dL/g. 350 mg of BUP &/or BUP HCl and 150 mg of PLGA powder were weighed and placed into a scintillation vial. The powders were mixed with an acoustic mixer. Implants were prepared in a die with a diameter of 3.5 mm and the die and punch were lightly coated with a magnesium stearate/acetone mixture. The mixture was placed in an oven at 125° C. for 1 hour, removed and subsequently compacted with a Carver press to 1000 pounds per square inch (PSI). The rods were ejected and allowed to equilibrate for 24 hours prior to characterization. Weights of the FB and HCl forms were held constant to minimize any differences of volume/weight differences due to the molecular weight differences of the FB and HCl forms. See Table 3.

TABLE 3

| | Buprenorphine FB | Buprenorphine HCl |
|---|---|---|
| Formulation | (mg) | (mg) |
| F1 - 100% FB | 350.0 | — |
| F2 - 66.7% FB/33.3% HCl | 233.4 | 116.6 |
| F3 - 33.3% FB/66.7% HCl | 116.6 | 233.4 |
| F4 - 100% HCl | — | 350.0 |

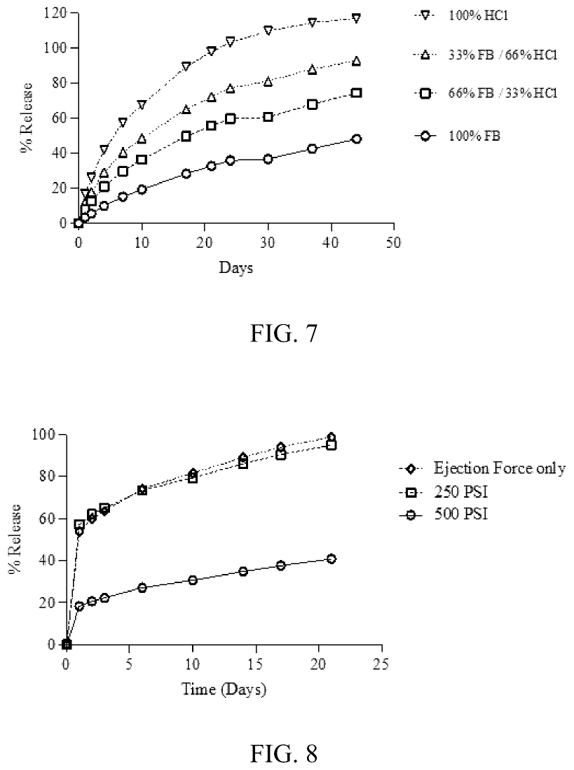

A comparison of the release rates of varying the ratio of the HCl:FB form in the implants was determined using the in vitro release methodology. Buprenorphine concentrations were determined through high performance liquid chromatography (HPLC). As shown in FIG. 7, the in vitro release rates of buprenorphine from the dosage forms is influenced by the ratio of the FB:HCl form, where higher free base concentrations slow the release rate with the same polymer matrix and similar drug loads and the release rate can be modified through mixing of solid-state forms of buprenorphine.

### Example 9: Implant Porosity from Powder Compaction

The porosity or free volume of implants can largely influence the release rate through a combination of including but not limited to the water uptake rate, drug diffusional rate, and biodegradation rate. By selecting an appropriate density/porosity, the release rate can be modified to obtain a preferable release rate for a clinically relevant duration of therapy.

Three types of implants were prepared using PLGA 75:25 (Durect, IV 0.55-0.75) 25-150 μm particles and buprenorphine FB particles less than 150 μm. Implants containing a total of 70% w/w buprenorphine were prepared. 350 mg of buprenorphine FB and 150 mg of PLGA powder were weighed and placed into a scintillation vial. The powders were then mixed with a Resodyn acoustic mixer. Implants were prepared in a die with a diameter of 3.5 mm and the die and punch were lightly coated with a magnesium stearate/acetone mixture. The mixture was placed in an oven at 125° C. for 1 hour, removed and subsequently compacted with a Carver press with either 500 or 250 PSI or compacted through the force via ejection only. The rods were then ejected and allowed to equilibrate for 24 hours prior to characterization.

As shown in FIG. 8, the release rate correlates to the compaction force/porosity of the implant, where the 500 PSI compacted rod has a slower release rate than the 250 PSI and rod compacted with the ejection force only.

### Example 10: Drug Loading Impact

Drug loading can impact the drug release. Buprenorphine was incorporated at a 50, 60, or 70% drug load in the dosage form using the FB form with a particle size of <150 μm and Corbion B54 PLGA particles as described in Example 4. 1% magnesium stearate was added to limit potential sticking to the die surface. Mixtures were loaded into a die with a diameter of 3.5 mm and placed into an oven at 100° C. for 1 hr. The die was removed and lightly compacted with a press. Post compaction, the die was immediately removed and allowed 24 hours to equilibrate to room temperature.

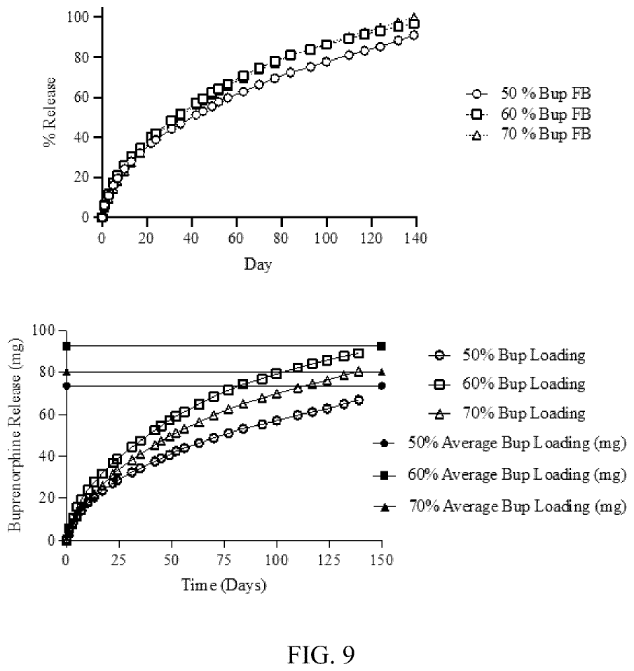

In vitro release comparisons were performed to demonstrate the impact drug loading can have on the in vitro release rate. FIG. 9 illustrates the in vitro release curves. In this example, similar release profiles were found for 60 and 70% buprenorphine FB loading, while the 50% loading showed a slightly slower release rate. The duration of in vitro release was greater than 90 days for each of the drug loadings tested.

What is claimed is:

1. A biodegradable implantable formulation comprising a drug and one or more biodegradable polymers, wherein the formulation is essentially free of pores.

2. The biodegradable formulation of claim 1, that comprises about 40 to 80% by weight of drug.

3. The biodegradable formulation of claim 1, that comprises about 50 to 70% by weight of drug.

4. The biodegradable formulation of claim 1, wherein the drug is buprenorphine.

5. The biodegradable formulation of claim 4, wherein the buprenorphine is of the free base form, salt form, or mixtures thereof.

6. The biodegradable formulation of claim 1, that comprises about 20 to 60% by weight of biodegradable polymer.

7. The biodegradable formulation of claim 1, that comprises about 30 to 50% by weight of biodegradable polymer.

8. The biodegradable formulation of claim 1, wherein the biodegradable polymer is at least one selected from the group consisting of poly(lactide-co-glycolide), Poly(D,L-lactide), Poly(ε-caprolactone), Polyhydroxybutyrate, Polyanhydrides, Polyorthoesters, and any combination of any of these.

9. The biodegradable formulation of claim 1, that provides sustained release of drug for about 90 days or longer.

10. The biodegradable formulation of claim 1, wherein a surface of the biodegradable formulation is free of pores.

11. A method of treating a subject with an opioid abuse disorder, the method comprising:
implanting in a subject a biodegradable implantable formulation comprising a drug that treats opioid abuse and one or more biodegradable polymers, wherein the formulation is essentially free of pores.

12. The method of claim 11, wherein the biodegradable formulation comprises about 40 to 80% by weight of drug.

13. The method of claim 11, wherein the biodegradable formulation comprises about 50 to 70% by weight of drug.

14. The method of claim 13, wherein the drug is buprenorphine.

15. The method of claim 14, wherein the buprenorphine is of the free base form, salt form, or mixtures thereof.

16. The method of claim 11, wherein the biodegradable formulation comprises about 20 to 60% by weight of biodegradable polymer.

17. The method of claim 11, wherein the biodegradable formulation comprises about 30 to 50% by weight of biodegradable polymer.

18. The method of claim 11, wherein the biodegradable polymer is at least one selected from the group consisting of poly(lactide-co-glycolide), Poly(D,L-lactide), Poly(ε-caprolactone), Polyhydroxybutyrate, Polyanhydrides, Polyorthoesters, and any combination of any of these.

19. The method of claim 11, wherein the biodegradable formulation provides sustained release of drug for about 90 days or longer.

20. A method of making a biodegradable formulation that is substantially free of pores, the method comprising:
providing a mixture of a drug and one or more biodegradable polymers;
compacting the mixture with sufficient mechanical force to remove surface pores and a substantial amount of internal pores; and
applying heat to sustainably remove remaining internal pores, thereby producing a biodegradable formulation that is substantially free of pores.
